(19) United States
(12) Patent Application Publication
(10) Pub. No.: US 2022/0135509 A1
(43) Pub. Date: May 5, 2022

(54) SYNTHETIC METHOD OF 9,9-BIS[4-(2-HYDROXYETHOXY)PHENYL]FLUORENE

(71) Applicant: Zhejiang Zhongxin Fluoride Materials Co., Ltd., SHAOXING (CN)

(72) Inventors: Qiliang YUAN, SHAOXING (CN); Dongdong JIANG, SHAOXING (CN); Ying WAN, SHAOXING (CN); Yonggen SHI, SHAOXING (CN); Pengfei XU, SHAOXING (CN); Haifeng CHEN, SHAOXING (CN)

(21) Appl. No.: 17/574,334

(22) Filed: Jan. 12, 2022

Related U.S. Application Data
(63) Continuation of application No. PCT/CN2021/092546, filed on May 10, 2021.

(30) Foreign Application Priority Data
Oct. 9, 2020 (CN) 202011071056.8

(51) Int. Cl. C07C 41/30; C07C 41/40

(52) U.S. Cl. CPC C07C 41/30; C07C 41/40

(57) ABSTRACT

A synthetic method of 9,9-bis[4-(2-hydroxyethoxy)phenyl]fluorene, belonging to the technical field of chemical synthesis. 9-fluorenone, phenoxyethanol, a catalyst and a cocatalyst are stiffed in an alkane solvent and heated until refluxing, the generated water is removed from the reaction solution via an azeotropic method while reacting, the reaction solution is diluted with water after the reaction is ended, uniformly stirred and cooled to separate out crystals and then filtered, a filter cake is rinsed and dried to obtain a 9,9-bis[4-(2-hydroxyethoxy)phenyl]fluorene finished product; the filtered crystallization mother liquor is subjected to standing and layering, a water phase is removed, then an organic phase is distilled to recycle the alkane solvent, and the concentrate is rectified to recycle phenoxyethanol.

### CROSS-REFERENCE TO RELATED APPLICATIONS

This application is a continuation of International Patent Application No. PCT/CN2021/092546 with a filing date of May 10, 2021, designating the United States, now pending, and further claims priority to Chinese Patent Application No. 202011071056.8 with a filing date of Oct. 9, 2020. The content of the aforementioned applications, including any intervening amendments thereto, are incorporated herein by reference.

### TECHNICAL FIELD

The disclosure belongs to the technical field of chemical synthesis, and particularly relates to a synthetic method of 9,9-bis [4-(2-hydroxyethoxy)phenyl]fluorene.

### BACKGROUD OF THE PRESENT INVENTION

9,9-bis[4-(2-hydroxyethoxy)phenyl]fluorene is an extremely important bisphenol compound. As a functional high polymer material monomer, it is mainly used for fabricating high polymer materials such as epoxy resins, polycarbonates, polyaromatic esters and polyethers having high thermal resistance, excellent optical performance and good flame retardance, and has a wide use in the fields of aerospaces, electronics, automobile manufacturing and the like. For example, polycarbonate synthesized by using 9,9-bis[4-(2-hydroxyethoxy)phenyl]fluorene as a monomer has excellent optical performance, and is used for manufacturing high-end resin cameras; epoxy resin synthesized by using 9,9-bis[4-(2-hydroxyethoxy)phenyl]fluorene as the monomer, as a packaging material, is widely applied to the fields of display screen fabricating, chip packaging and the like. Therefore, with the advent of the 5G era, the Internet of Everything has become a reality, and 9,9-bis[4-(2-hydroxyethoxy)phenyl]fluorene as a basic support material will bring a golden period of rapid development.

There two synthetic routes of 9,9-bis[4-(2-hydroxyethoxy)phenyl]fluorene:

Route I: 9,9-bis[4-(2-hydroxyethoxy)phenyl]fluorene is used as a raw material to react with ethylene glycol carbonate or ethylene oxide or 2-haloethanol or the like to synthesize 9,9-bis[4-(2-hydroxyethoxy)phenyl]fluorene:

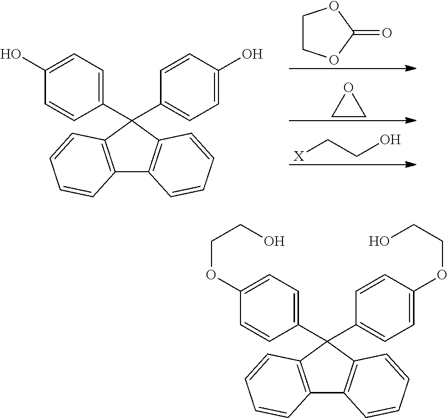

This synthetic route has the advantages of simple reaction, high synthesis yield, few three wastes and the like, but the raw material 9,9-bis[4-(2-hydroxyethoxy)phenyl]fluorene is expensive in price and unavailable to result in high synthesis cost and poor market competitiveness, and therefore has been gradually eliminated at present.

Route II: 9-fluorenone is used as a raw material to react with phenoxyethanol under the combined action of a strong acidic catalyst and a thiol compound cocatalyst to synthesize 9,9-bis[4-(2-hydroxyethoxy)phenyl]fluorene:

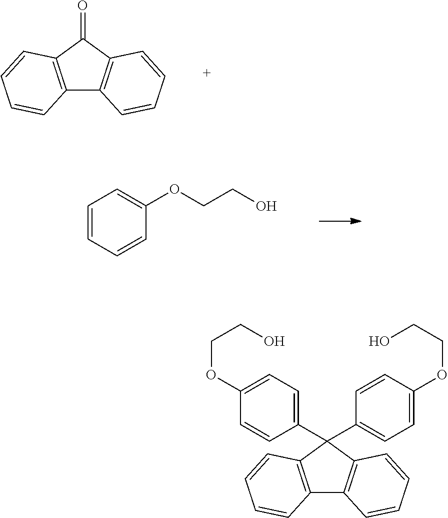 [SMILES: O=C1c2ccccc2-c2ccccc21 ; OCCOc1ccccc1 ; OCCOc1ccc(C2(c3ccc(OCCO)cc3)c3ccccc3-c3ccccc32)cc1]

This synthetic route has the advantages of cheap and available raw material, simple reaction, high synthesis yield and the like. However, it is needed to use a large amount of strong acidic catalysts such as concentrated sulfuric acid, hydrogen chloride, solid heteropolyacid and superacid in the synthesis process so as to generate a plenty of acidic wastes, resulting in a large environmental protection pressure.

### SUMMARY OF PRESENT INVENTION

The objective of the disclosure is to provide an industrial synthetic method of 9,9-bis[4-(2-hydroxyethoxy)phenyl]fluorene. Since the synthetic route II of 9,9-bis[4-(2-hydroxyethoxy)phenyl]fluorene has stronger competitiveness compared with the route I, the disclosure is based on the synthetic route II, and the following optimizations are realized through process conditions: 1) use of a large amount of strong acids is avoided, a catalytic amount of strong acid is only used, so as to realize the efficient synthesis of 9,9-bis[4-(2-hydroxyethoxy)phenyl]fluorene, thereby greatly reducing the generation and emission of acidic wastes; 2) the reaction solvent and phenoxyethanol that does not participate in the reaction are recycled from the mother liquor via distillation, rectification and other manners, thereby reducing synthesis cost. The disclosure has the advantages of cheap and available raw materials, simple operation, good atomic economy, high synthesis yield, good product quality, environmental friendliness and the like, and is suitable for industrial application.

The technical solution adopted by the disclosure is as follows:

Provided is a synthetic method of 9,9-bis[4-(2-hydroxyethoxy)phenyl]fluorene, comprising the following steps:

(1) stirring and heating 9-fluorenone, phenoxyethanol, a catalyst and a cocatalyst in an alkane solvent until refluxing, and removing the generated water from the reaction solution via an azeotropic method while reacting to obtain a mixed solution containing 9,9-bis[4-(2-hydroxyethoxy)phenyl]fluorene;

(2) diluting the mixed solution containing 9,9-bis[4-(2-hydroxyethoxy)phenyl]fluorene with water, uniformly stirring and cooling to separate out crystals, filtering, rinsing a filter cake and drying to obtain a 9,9-bis[4-(2-hydroxyethoxy)phenyl]fluorene finished product; and

(3) standing and layering the obtained crystallization mother liquor, removing a water phase, then distilling an organic phase to recycle the alkane solvent, and rectifying the concentrate to recycle phenoxyethanol.

The technical route adopted by the disclosure can be represented by the following reaction formula:

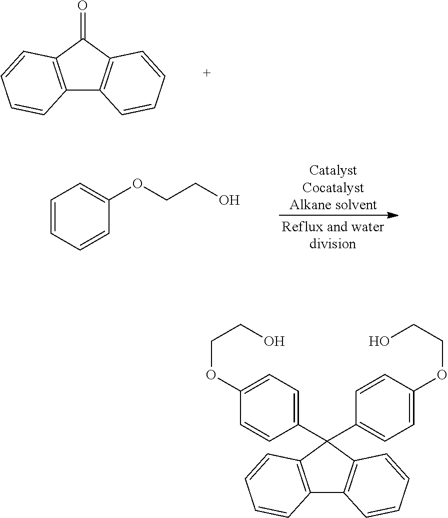 [SMILES: O=C1c2ccccc2-c2ccccc21 ; OCCOc1ccccc1 ; OCCOc1ccc(C2(c3ccc(OCCO)cc3)c3ccccc3-c3ccccc32)cc1]

The disclosure is further set below:

9-fluorenone and phenoxyethanol are used as main reaction raw materials. It can be seen from the reaction equation that 1 equivalent of 9-fluorenone needs to react with 2 equivalents of phenoxyethanol to obtain 9,9-bis[4-(2-hydroxyethoxy)phenyl]fluorene. Therefore, the minimum molar ratio of 9-fluorenone to phenoxyethanol is 2:1. To accelerate the reaction speed and simultaneously ensure that 9-fluorenone sufficiently participates in the reaction, excessive phenoxyethanol is usually used, however, use of more and more phenoxyethanol does not cause a good result, excessive phenoxyethanol cannot promote the reaction speed but rather causes reduction in the reaction speed due to dilution of concentrations of dilution catalysts and cocatalysts, preferably, a molar ratio of 9-fluorenone to phenoxyethanol is 1:(2-6).

The catalyst means a strong acidic compound, which can be an organic acidic compound or an inorganic acidic compound, and is selected from one or more of concentrated sulfuric acid, methanesulfonic acid, benzenesulfonic acid, p-toluenesulfonic acid, heteropoly acid, superacid, solid acid, hydrogen halide and the like, and a more preferred catalyst is one or two of concentrated sulfuric acid and methanesulfonic acid. A mass ratio of catalyst to 9-fluorenone is (0.0001-1):1, preferably, the mass ratio of catalyst to 9-fluorenone is (0.0005-0.5):1, more preferably, the mass ratio of catalyst to 9-fluorenone is (0.001-0.2):1.

The cocatalyst means a linear alkyl carboxylic acid and alkyl alcohol compound containing a mercapto group in a molecular structure, and can be represented by the following general formula:

HS—(CH₂)n-CO₂H _{and} HS—(CH₂)m—OH

wherein, n is 1-9, and m is 2-10. The cocatalyst can be a single mercapto-containing linear alkyl carboxylic acid or a mixture of a plurality of mercapto-containing linear alkyl carboxylic acids; a single mercapto-containing linear alkyl alcohol or a mixture of a plurality of mercapto-containing linear alkyl alcohols; or a mixture of a mercapto-containing linear alkyl carboxylic acid and a mercapto-containing linear alkyl alcohol. The cocatalyst is selected from one or more of mercaptoacetic acid, 3-mercaptopropionic acid, 4-mercaptobutyric acid, 5-mercaptovaleric acid, 6-mercaptohexanoic acid, 7-mercaptoheptanoic acid, 8-mercaptooctanoic acid, 9-mercaptononanoic acid, 10-mercaptodecanoic acid, ethylmercaptan, 3-mercaptopropanol, 4-mercaptobutanol, 5-mercaptopentanol, 6-mercaptohexanol, 7-mercaptoheptanol, 8-mercaptooctanol, 9-mercaptononanol and 10-mercaptodecane alcohol. A mass ratio of cocatalyst to 9-fluorenone is (0.0001-0.2):1, preferably, a mass ratio of cocatalyst to 9-fluorenone is (0.001-0.1):1.

The alkane solvent means C₆-Cio linear, branched or cyclic alkane. Compared with the traditional aromatic hydrocarbon solvent, such as toluene, xylene and chlorobenzene, the selected alkane solvent has the advantages that in the process of reaction, the alkane solvent cannot participate in the reaction and side reactions do not occur, but the traditional aromatic hydrocarbon solvent together with raw material 9-fluorenone and a strong acidic catalyst can have side reactions such as Friedel-Crafts reaction and sulfonation reaction, thereby causing reduction in reaction yield and decrease in product quality. The selection of the alkane solvent is mainly related to its boiling point and azeotropy with water. If the boiling point of the alkane solvent is too low, the temperature of reflux reaction is too low, the reaction speed is too slow, which is disadvantageous to improvement of synthesis efficiency; if the boiling point of the alkane solvent is too high, the temperature of reflux reaction is too high, side reactions are increased, thereby causing reduction in reaction yield and product quality. In addition to selecting a proper boiling point, the azeotropic performance of the alkane solvent with water is crucial, if the alkane solvent cannot form effective azeotrope with water, water generated by the reaction cannot be timely removed from a reaction system by azeotropically entraining water, which is disadvantageous to proceeding of the reaction. The preferred alkane solvent can be one or more of n-hexane, n-heptane, n-octane, isooctane, nonane, decane, cyclohexane, methylcyclohexane and ethylcyclohexane. The amount of the alkane solvent is (0.1-10) times the mass of 9-fluorenone, preferably, the amount of the alkane solvent is 0.3-5 times the mass of 9-fluorenone.

The reaction process is as follows:

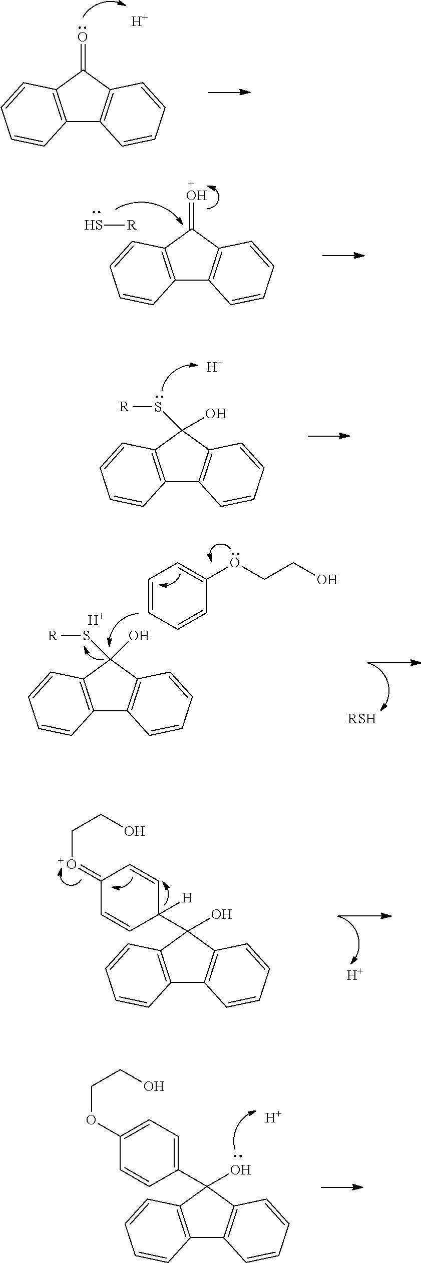

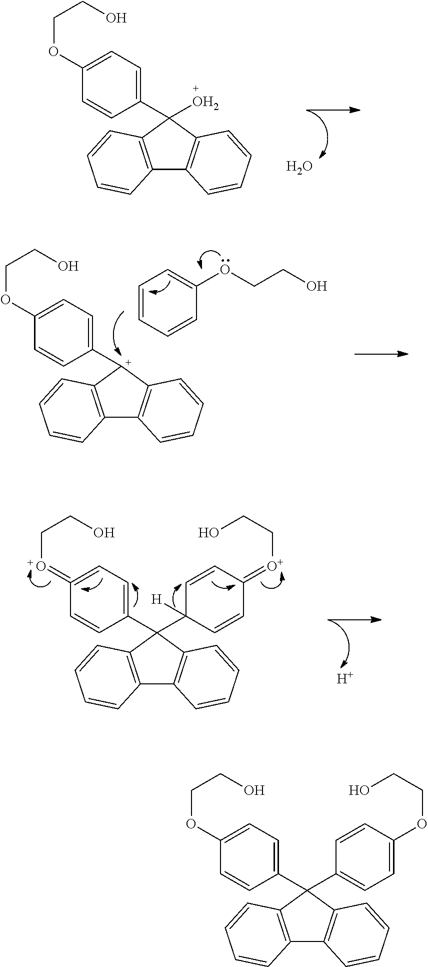

The 9,9-bis[4-(2-hydroxyethoxy)phenyl]fluorene mixed solution obtained after the reaction is ended has viscous physical property, which is not conducive to solid-liquid separation. Through dilution with water, the property of the reaction solution can be effectively improved, solid-liquid separation after cooling to separate out crystals is facilitated, and the amount of water is (0.1-10) times the mass of 9-fluorenone, preferably, the amount of water is (0.1-3) times the mass of 9-fluorenone.

The reaction solution after being diluted with water is gradually cooled to room temperature or a lower temperature, a preferred temperature is 0-30° C., the cooled reaction solution is stirred to separate out crystals for 0-5 h, and solid-liquid separation is performed after the crystals are sufficiently separated out. The solid obtained by separation is rinsed with pure water until the pH of the rinsing solution is neutral, and the wet product is dried to obtain a 9,9-bis[4-(2-hydroxyethoxy)phenyl]fluorene finished product with a content of ≥99.0% and a yield of >90%.

The crystallization mother liquor is subjected to standing and layering to remove a water phase, and the main components of the obtained organic phase are an alkane solvent and unreacted phenoxyethanol which have a high recycle value. The organic phase is distilled to recycle the alkane solvent which has a recovery rate of more than 90% and a content of more than 99%, and then rectified to recycle phenoxyethanol which has a recovery rate of more than 90% and a content of more than 99%. The recycled alkane solvent and phenoxyethanol are both used for synthesizing 9,9-bis[4-(2-hydroxyethoxy)phenyl]fluorene without affecting the product quality and reaction yield.

Compared with the prior art, the disclosure has the beneficial effects:

1. The alkane solvent is used to replace the traditional aromatic hydrocarbon solvent, thereby avoiding that the solvent participates in side reaction and improving the reaction yield and product quality;

2. Water generated in the reaction is brought out from the system by using an azeotropy and dehydration manner so as to ensure the smooth proceeding of the reaction while greatly reducing the amount of the acidic catalyst, and the generation amount of acidic wastes is significantly reduced, and the process is friendly to the environment;

3. The reaction solvent and unreacted phenoxyethanol are recycled, thereby improving atomic economy, reducing production cost and decreasing the emission of three wastes;

4. The disclosure has the advantages of cheap and available raw materials, simple operation, good atomic economy, high synthesis yield, good product quality, environmental friendliness and the like, and is suitable for industrial application.

The disclosure will be further illustrated in combination with specific embodiments below.

### DETAILED DESCRIPTION OF PREFERRED EMBODIMENTS

### Example 1

180 g of 9-fluorenone, 415 g of phenoxyethanol, 1.8 g of 3-mercaptopropionic acid and 540 g of cyclohexane were added into a 2 L reaction bottle. The above raw materials were stirred, and 27 g of concentrated sulfuric acid was dropwise added. After dropwise addition was ended, the temperature was raised until refluxing, refluxing and water division was conducted for 24 h while reacting, and the reaction was stopped. 360 g of water was added, the temperature was reduced to separate out crystals, the above reaction solution was stirred for 2 h at 20-30° C. and filtered, a filter cake was rinsed with pure water until the pH of the rinsing solution was neutral, and the filter cake was dried to obtain 412.2 g of 9,9-bis[4-(2-hydroxyethoxy)phenyl]fluorene with a yield of 94.1% and a content of 99.2%.

The crystallization mother liquor obtained after filtration was subjected to standing to remove a water phase, and an organic phase was distilled at normal pressure to obtain 492g of cyclohexane with a content of 99.7% and a recovery rate of 91.1%. The concentrated solution was rectified at reduced pressure to obtain 127.1 g of phenoxyethanol with a content of 99.2%. After the theoretical consumption was deducted, the recovery rate was 91.5%.

### Example 2

90 g of 9-fluorenone, 173 g of phenoxyethanol, 1.8 g of mercaptoacetic acid, 315 g of n-heptane and 9 g of methylsulphonic acid were added into a 1 L reaction bottle, stirred and heated until refluxing, refluxing and water division was conducted for 18 h while reacting, and the reaction was stopped. 135 g of water was added, the temperature was reduced to separate out crystals, the above reaction solution was stirred for 2 h at 0-10° C. and filtered, a filter cake was rinsed with pure water until the pH of the rinsing solution was neutral, and the filter cake was dried to obtain 203.2 g of 9,9-bis[4-(2-hydroxyethoxy)phenyl]fluorene with a yield of 92.8% and a content of 99.1%.

The crystallization mother liquor obtained after filtration was subjected to standing to remove a water phase, and an organic phase was distilled at normal pressure to obtain 291.4 g of n-heptane with a content of 99.8% and a recovery rate of 92.5%. The concentrated solution was rectified at reduced pressure to obtain 31.6 g of phenoxyethanol with a content of 99.1%. After the theoretical consumption was deducted, the recovery rate was 90.4%.

### Example 3

135 g of 9-fluorenone, 465 g of phenoxyethanol, 0.7 g of 8-mercaptooctanol, 200 g of methyl cyclohexane and 7 g of concentrated sulfuric acid were added into a 2 L reaction bottle, stirred and heated until refluxing, refluxing and water division was conducted for 20 h while reacting, and the reaction was stopped. 135 g of water was added, the temperature was reduced to separate out crystals, the above reaction solution was stirred for 3 h at 10-15° C. and filtered, a filter cake was rinsed with pure water until the pH of the rinsing solution was neutral, and the filter cake was dried to obtain 310.1 g of 9,9-bis[4-(2-hydroxyethoxy)phenyl]fluorene with a yield of 94.4% and a content of 99.2%.

The crystallization mother liquor obtained after filtration was subjected to standing to remove a water phase, and an organic phase was distilled at normal pressure to obtain 181.8 g of methyl cyclohexane with a content of 99.6% and a recovery rate of 90.9%. The concentrated solution was rectified at reduced pressure to obtain 242.7 g of phenoxyethanol with a content of 99.6%. After the theoretical consumption was deducted, the recovery rate was 94.1%.

### Example 4

100 g of 9-fluorenone, 300 g of phenoxyethanol, 3 g of 3-mercaptopropanol, 1 g of concentrated sulfuric acid and 200 g of isooctane were added into a 1 L reaction bottle, stirred and heated until refluxing, refluxing and water division was conducted for 15 h while reacting, and the reaction was stopped. 50 g of water was added, the temperature was reduced to separate out crystals, the above reaction solution was stirred for 1 h at 20-25° C. and filtered, a filter cake was rinsed with pure water until the pH of the rinsing solution was neutral, and the filter cake was dried to obtain 228.3 g of 9,9-bis[4-(2-hydroxyethoxy)phenyl]fluorene with a yield of 93.8% and a content of 99.4%.

The crystallization mother liquor obtained after filtration was subjected to standing to remove a water phase, and an organic phase was distilled at normal pressure to obtain 184.6 g of isooctane with a content of 99.7% and a recovery rate of 92.3%. The concentrated solution was rectified at reduced pressure to obtain 136.8 g of phenoxyethanol with a content of 99.7%. After the theoretical consumption was deducted, the recovery rate was 93.3%.

### Example 5

150 g of 9-fluorenone, 400 g of phenoxyethanol, 7.5 g of ethylmercaptan, 375 g of ethyl cyclohexane and 4.5 g of concentrated sulfuric acid were added into a 2 L reaction bottle, stirred and heated until refluxing, refluxing and water division was conducted for 12 h while reacting, and the reaction was stopped. 120 g of water was added, the temperature was reduced to separate out crystals, the above reaction solution was stirred for 2 h at 0-5° C. and filtered, a filter cake was rinsed with pure water until the pH of the rinsing solution was neutral, and the filter cake was dried to obtain 344.9 g of 9,9-bis[4-(2-hydroxyethoxy)phenyl]fluorene with a yield of 94.5% and a content of 99.3%.

The crystallization mother liquor obtained after filtration was subjected to standing to remove a water phase, and an organic phase was distilled at normal pressure to obtain 343.9 g of ethyl cyclohexane with a content of 99.5% and a recovery rate of 91.7%. The concentrated solution was rectified at reduced pressure to obtain 157.7 g of phenoxyethanol with a content of 99.4%. After the theoretical consumption was deducted, the recovery rate was 92.8%.

### Example 6

80 g of 9-fluorenone, 300 g of phenoxyethanol, 1.2 g of 6-mercaptohexanoic acid, 6.4 g of methanesulfonic acid and 80 g of n-octane were added into a 1 L reaction bottle, stirred and heated until refluxing, refluxing and water division was conducted for 12 h while reacting, and the reaction was stopped. 100 g of water was added, the temperature was reduced to separate out crystals, the above reaction solution was stirred for 3 h at 15-20° C. and filtered, a filter cake was rinsed with pure water until the pH of the rinsing solution was neutral, and the filter cake was dried to obtain 181.6 g of 9,9-bis[4-(2-hydroxyethoxy)phenyl]fluorene with a yield of 93.3% and a content of 99.5%.

The crystallization mother liquor obtained after filtration was subjected to standing to remove a water phase, and an organic phase was distilled at normal pressure to obtain 72.4 g of n-octane with a content of 99.3% and a recovery rate of 90.5%. The concentrated solution was rectified at reduced pressure to obtain 166.5 g of phenoxyethanol with a content of 99.5%. After the theoretical consumption was deducted, the recovery rate was 93.9%.

### Example 7

120 g of 9-fluorenone, 320 g of recycled phenoxyethanol, 1.2 g of 3-mercaptopropionic acid, 150 g of recycled n-heptane and 6 g of concentrated sulfuric acid were added into a 1 L reaction bottle, stirred and heated until refluxing, refluxing and water division was conducted for 20 h while reacting, and the reaction was stopped. 100 g of water was added, the temperature was reduced to separate out crystals, the above reaction solution was stirred for 2 h at 10-15° C. and filtered, a filter cake was rinsed with pure water until the pH of the rinsing solution was neutral, and the filter cake was dried to obtain 273 g of 9,9-bis[4-(2-hydroxyethoxy)phenyl]fluorene with a yield of 93.5% and a content of 99.4%.

The crystallization mother liquor obtained after filtration was subjected to standing to remove a water phase, and an organic phase was distilled at normal pressure to obtain 138.3 g of n-octane with a content of 99.3% and a recovery rate of 92.2%. The concentrated solution was rectified at reduced pressure to obtain 126.6 g of phenoxyethanol with a content of 99.5%. After the theoretical consumption was deducted, the recovery rate was 93.1%.

### Comparative Example

60 g of 9-fluorenone, 140 g of phenoxyethanol, 1 g of 3-mercaptopropionic acid, 6 g of concentrated sulfuric acid and 150 g of n-heptane were added into a 500 mL reaction bottle, stirred and heated until refluxing, refluxing and water division was conducted for 25 h while reacting, a sampling was conducted for HPLC detection, the content of the product was 97.9%, and the reaction was stopped. 60 g of water was added, the temperature was reduced to separate out crystals, the above reaction solution was stirred for 3 h at 0-10° C. and filtered, a filter cake was rinsed with pure water until the pH of the rinsing solution was neutral, and the filter cake was dried to obtain 135 g of 9,9-bis[4-(2-hydroxyethoxy)phenyl]fluorene with a yield of 92.5% and a content of 99.2%.

60 g of 9-fluorenone, 140 g of phenoxyethanol, 1 g of 3-mercaptopropionic acid, 6 g of concentrated sulfuric acid and 80 g of toluene were added into a 500 mL reaction bottle, stirred and heated until refluxing, refluxing and water division was conducted for 25 h while reacting, sampling was conducted for HPLC detection, the content of the product was 89.6%, and the reaction was stopped. 60 g of water was added, the temperature was reduced to separate out crystals, the above reaction solution was stirred for 3 h at 0-10° C. and filtered, a filter cake was rinsed with pure water until the pH of the rinsing solution was neutral, and the filter cake was dried to obtain 120.2 g of 9,9-bis[4-(2-hydroxyethoxy)phenyl]fluorene with a yield of 82.3% and a content of 94.9%.

60 g of 9-fluorenone, 140 g of phenoxyethanol, 1 g of 3-mercaptopropionic acid and 6 g of concentrated sulfuric acid were added into a 500 mL reaction bottle, then stirred and heated to 110° C. to react for 25 h, sampling was conducted for HPLC detection, the content of the product 9,9-bis[4-(2-hydroxyethoxy)phenyl]fluorene was 38.2%, and the reaction was stopped.

What is claimed is:

1. A synthetic method of 9,9-bis[4-(2-hydroxyethoxy)phenyl]fluorene, comprising the following steps:
stirring and heating 9-fluorenone, phenoxyethanol, a catalyst and a cocatalyst in an alkane solvent until refluxing, and removing the generated water from the reaction solution via an azeotropic method while reacting to obtain a mixed solution containing 9,9-bis[4-(2-hydroxyethoxy)phenyl]fluorene; wherein
the catalyst is selected from one or two of concentrated sulfuric acid and methanesulfonic acid, and a mass ratio of the catalyst to 9-fluorenone is 0.001-0.2:1;
the cocatalyst means a linear alkyl carboxylic acid and alkyl alcohol compound containing a mercapto group in a molecular structure, and is selected from one or more of mercaptoacetic acid, 3-mercaptopropionic acid, 4-mercaptobutyric acid, 5-mercaptovaleric acid, 6-mercaptohexanoic acid, 7-mercaptoheptanoic acid, 8-mercaptooctanoic acid, 9-mercaptononanoic acid, 10-mercaptodecanoic acid, ethylmercaptan, 3-mercaptopropanol, 4-mercaptobutanol, 5-mercaptopentanol, 6-mercaptohexanol, 7-mercaptoheptanol, 8-mercaptooctanol, 9-mercaptononanol and 10-mercaptodecane alcohol, and a mass ratio of cocatalyst to 9-fluorenone is 0.001-0.1:1;
the alkane solvent is selected from one or more of n-hexane, n-heptane, n-octane, isooctane, nonane, decane, cyclohexane, methylcyclohexane and ethylcyclohexane;
diluting the mixed solution containing 9,9-bis[4-(2-hydroxyethoxy)phenyl]fluorene with water, uniformly stirring and cooling to separate out crystals, filtering, rinsing a filter cake and drying to obtain a 9,9-bis[4-(2-hydroxyethoxy)phenyl]fluorene finished product; and
standing and layering the obtained crystallization mother liquor, removing a water phase, then distilling an organic phase to recycle the alkane solvent, and rectifying the concentrate to recycle phenoxyethanol.

2. The synthetic method of 9,9-bis[4-(2-hydroxyethoxy)phenyl]fluorene according to claim 1, wherein a molar ratio of 9-fluorenone to phenoxyethanol is 1:2-6.

3-5. (canceled)

6. The synthetic method of 9,9-bis[4-(2-hydroxyethoxy)phenyl]fluorene according to claim 1, wherein the amount of the alkane solvent is [[0.3-5]]0.1-10 times the mass of 9-fluorenone.

7. The synthetic method of 9,9-bis[4-(2-hydroxyethoxy)phenyl]fluorene according to claim 1, wherein the amount of water for dilution is 0.1-3 times the mass of 9-fluorenone.
